(19) United States
(12) Patent Application Publication
(10) Pub. No.: US 2017/0224605 A1
(43) Pub. Date: Aug. 10, 2017

(54) TEMPORARY CHANGING THE COLOR OF HAIR USING PIGMENTS, ALCOHOLS AND CATIONIC CELLULOSES

(71) Applicant: Henkel AG & Co. KGaA, Duesseldorf (DE)

(72) Inventors: Konstantin Goutsis, Juechen (DE); Gabriele Weser, Neuss (DE)

(73) Assignee: Henkel AG & Co. KGaA, Duesseldorf (DE)

(21) Appl. No.: 15/494,383

(22) Filed: Apr. 21, 2017

Related U.S. Application Data
(63) Continuation of application No. PCT/EP2015/071723, filed on Sep. 22, 2015.

(30) Foreign Application Priority Data
Oct. 23, 2014 (DE) 10 2014 221 533.1

(51) Int. Cl. A61K 8/86; A61K 8/365; A61K 8/41; A61Q 5/06; A61K 8/73; A61K 8/25; A61K 8/29; A61K 8/19; A61K 8/44; A61K 8/34

(52) U.S. Cl. CPC A61K 8/86; A61K 8/44; A61K 8/365; A61K 8/416; A61K 8/345; A61K 8/731; A61K 8/34; A61K 8/25; A61K 8/29; A61K 8/19; A61Q 5/065; A61Q 5/06

(57) ABSTRACT

An agent for temporarily changing the color of keratin fibers, and in particular of human hair, includes, in an aqueous cosmetic carrier, at least one aliphatic and/or aromatic alcohol having 2 to 8 carbon atoms, at least one color pigment, and at least one cationic cellulose. The total amount of the lipids present in the agent, based on the total weight of the agent, is less than 2.5% by weight.

### FIELD OF THE INVENTION

The present invention generally relates to agents for temporarily changing the color of keratin fibers, in particular human hair, which are based on an aqueous-alcoholic carrier and comprise color pigments and cationic celluloses. The present invention furthermore relates to a method for changing the color of and for styling hair, wherein a corresponding agent is sprayed onto the hair and, at the same time, the hair is styled.

### BACKGROUND OF THE INVENTION

Changing the shape and color of keratin fibers, and in particular of hair, represents an important field of modern cosmetics. For changing the color of hair, a person skilled in the art knows a variety of dyeing systems, depending on the requirements in the regard to the coloration. Usually, oxidation dyes are used for permanent, intensive colorations having good fastness properties and good gray coverage. Such coloring agents usually comprise oxidation dye precursors, so-called developer components and coupler components, which create the actual dyes among each other under the influence of oxidizing agents, such as hydrogen peroxide. Oxidation dyes are characterized by very long lasting dyeing results.

When direct dyes are used, dyes that have already fully formed diffuse from the coloring agent into the hair color. Compared to oxidative hair dyeing, the colorations obtained with direct dyes do not last as long and wash out more quickly. Colorations by way of direct dyes usually remain on the hair for a time period between 5 and 20 shampoos.

Within the scope of modern fashion trends, there is also a need for color effects that remain on the hair for a short period of time and can subsequently be completely removed again from the hair, without residue, by washing the hair. Direct dyes diffuse substantially strongly into the hair fiber, where they last for several hair washes; this class of dye, however, is not suited well for the residue-free removal of the color effect.

The use of color pigments is known to achieve short-lived color changes in the hair. Color pigments are generally understood to mean insoluble, color-imparting substances. These are present undissolved in the dye formulation in the form of small particles, and these particles deposit on the hair fiber only from the outside. They remain there until the next time the hair is washed and can be removed without residue by way of shampooing. A variety of products of this type are available in the market under the name hair mascara.

Since it is possible to remove hair mascaras by washing the hair, these are generally designed as “leave-on” products. It is particularly advantageous for users of a “leave-on” product if they are able to carry out slight temporary shaping of the hair simultaneously with temporarily changing the color. Temporary shaping includes, for example, styling such as curling, straightening, teasing or also setting. Temporary shaping can be achieved by way of styling agents, for example, such as hair sprays, hair waxes, hair gels, hair setting lotions, blow drying lotions, styling sprays and the like Temporary shaping is also referred to as hair styling or styling, and shaping agents are also referred to as styling agents.

Products that allow a color and shape change at the same time are already known from the prior art. For example, WO 9920230 A2 describes hair mascara products, comprising pigments together with non-ionic polymers and waxes having a high melting point.

WO 2014146818 A1 also discloses styling agents comprising pigments that are characterized by the presence of solid fatty alcohols and waxes.

The fat and wax components included in these products are usually provided to establish a certain drying time, which gives the consumer the sensation of dry hair, while the mascara applied to the hair maintains a residual moisture level which ensures that the hair style can still be shaped and combed.

Especially products that remain on the hair, however, are often associated with the problem that the hair is weighed down by the presence of the lipid bodies. Visually, this creates the impression of “greasy hair,” and the setting properties of these products are comparatively poor. Combination products for changing the color and for styling therefore continue to offer room for improvement.

Accordingly, it is desirable to provide a hair mascara product that is versatile in its use and allows the color of hair to be changed temporarily. It should be possible to formulate the hair mascara such that the same can be applied by way of a sponge, via a brush, or by way of a spray application. The change in color should take place easily and with minimal damage and should be removable from the hair again, without residue, by washing. During the time frame until the next time the hair is washed, however, the product present on the hair should be extremely resistant to external conditions, which is to say no loss of color or any other separation of the product should become apparent, neither due to abrasion on textiles nor as a result of combing. At the same time, the hair having been dyed in this way should have a soft feel, not be weighed down, not feel rough or greasy, and also visually not leave the impression of greasy hair. Furthermore, other desirable features and characteristics of the present invention will become apparent from the subsequent detailed description of the invention and the appended claims, taken in conjunction with this background of the invention.

### BRIEF SUMMARY OF THE INVENTION

A first subject matter of the present invention is an agent for temporarily changing the color of keratin fibers, and in particular of human hair, comprising, in an aqueous cosmetic carrier,

(a) at least one aliphatic and/or aromatic alcohol having 2 to 8 carbon atoms;
(b) at least one color pigment; and
(c) at least one cationic cellulose,
wherein the total amount of the lipids (d) present in the agent, based on the total weight of the agent, is less than 2.5% by weight.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has been shown that these objects can be achieved by using color pigments and certain non-ionic, polyethoxylated silicones if these are used in a special aqueous-alcoholic carrier, which is characterized by a low content of lipid bodies.

Keratinic fibers, keratin-containing fibers or keratin fibers shall be understood to mean furs, wool, feathers, and in particular human hair. While the agents according to the invention are primarily suitable for lightening and dyeing keratin fibers, in principle there are no objections to using them in other fields as well.

The term “temporarily changing the color” within the scope of the present invention shall be understood to mean a temporary coloration of the hair that can be completely, or substantially completely, removed by washing the hair (using a commercially available shampoo). The term “temporarily changing the color” within the meaning of the present invention does not cover oxidative dyeing carried out by way of oxidation dyes. The term “temporarily changing the color” likewise does not cover any lightening, bleaching or blonding of keratin fibers caused by the use of an oxidizing agent. Both the effect caused by oxidative dyeing and the effect caused by blonding cannot be reversed by washing the hair, and therefore neither color change is temporary.

The agents comprise each of the ingredients that are essential to the invention in an aqueous cosmetic carrier. For the purpose of temporarily changing the color and shape, such carriers can be gels or surfactant-containing foaming solutions, for example, such as shampoos, sprayable solutions, foam aerosols or foam formulations.

As a first ingredient (a) that is essential to the invention, the agents according to the invention comprise at least one aliphatic and/or aromatic alcohol having 2 to 8 carbon atoms. Aliphatic and/or aromatic alcohols having 2 to 8 carbon atoms are compounds that comprise 2 to 8 carbon atoms, are of an aliphatic and/or aromatic nature, and carry one or more hydroxy groups.

The alcohols (a) within the meaning of the present invention do not carry any heteroatoms other than oxygen. They can comprise an ether grouping, but beyond that do not comprise any functional groups other than the hydroxy group (which is to say, monoethanolamine, alpha-hydroxycarboxylic acids, dihydroxyacetone, and the like, are not alcohols within the meaning of the present invention).

Suitable aliphatic alcohols are, for example, ethanol, isopropanol, n-propanol, butanol, n-pentanol, 1,2-propanediol, 1,3-propandiol, 1,3-butanediol, 1,4-butanediol, 1,2-hexanediol, 1,6-hexanediol and glycerol. Suitable aromatic alcohols are benzyl alcohol, phenoxyethanol and phenylethyl alcohol, for example.

In one embodiment, an agent according to the invention for temporarily changing the color of keratin fibers is characterized by comprising one or more alcohols (a) from the group consisting of ethanol, isopropanol, n-propanol, butanol, n-pentanol, 1,2-propanediol, 1,3-propandiol, 1,3-butanediol, 1,4-butanediol, 1,2-hexanediol, 1,6-hexanediol, glycerol, benzyl alcohol, phenoxyethanol and phenylethyl alcohol.

The alcohols (a) according to the invention are organic solvents that contribute to the dissolution of the cationic cellulose (c) and, after the agent has been applied to the keratin fibers, influence the film formation rate of cationic celluloses (c). It has been found that this film formation takes place particularly well and particularly evenly when the alcohol is, or the alcohols are, present in the agent in a minimum amount of at least 10.0 wt. %. The best results were observed at an alcohol amount of at least 40 wt. %. All information in wt. % is based on the total amount of all alcohols (a) according to the invention relative to the total weight of the agent.

In a preferred embodiment, an agent according to the invention is characterized by comprising, based on the total weight thereof, one or more alcohols (a) in a total amount of at least 10.0 wt. %, preferably of at least 20.0 wt. %, more preferably of at least 30.0 wt. %, still more preferably of at least 40.0 wt. %, and especially particularly preferably of at least 50.0 wt. %.

The alcohols from group (a) are compounds having varying boiling points and varying levels of volatility. It has been found that, within this group, ethanol is suited the best. If the agents comprise significant amounts of ethanol, the pigments deposited on the keratin fiber are enclosed by a polymer film of the cationic celluloses (c), which is designed such that the pigments adhere to the keratin fiber particularly well. In this case, the color result is particularly even, and the abrasion of the pigments caused by friction on textiles is minimized.

In an especially particularly preferred embodiment, an agent according to the invention is thus characterized by comprising, based on the total weight thereof, at least 15.0 wt. %, preferably at least 25.0 wt. %, more preferably at least 35.0 wt. %, and especially particularly preferably at least 45.0 wt. % ethanol.

The above-described properties can be improved even further if a small amount of a further polyhydric alcohol having low volatility is added to the ethanol, for example 1,2-propanediol or glycerol.

In a further especially particularly preferred embodiment, an agent according to the invention is thus characterized by comprising, based on the total weight thereof—1,2-propanediol and/or glycerol in a total amount of 0.1 to 7.0 wt. %, preferably 0.5 to 5.5 wt. %, more preferably 1.0 to 3.5 wt. %, and particularly preferably at 1.5 wt. % to 2.5 wt. %. The agents according to the invention comprise all essential components in an aqueous carrier. Due to the water content of the agent, it is likewise possible to influence the deposition of the pigments on the keratin fibers and the film formation of the cationic celluloses (c). If the water content is too high, there is a risk that the product does not dry sufficiently quickly. In particular, when the agents are set to a lower viscosity (for example, because they are to be sprayed), the color result may turn out more unevenly. In this connection, a water content between 20 and 60 wt. %, preferably between 24 and 54 wt. %, more preferably between 28 and 50 wt. %, and particularly preferably between 32 and 42 wt. % has proven to be well-suited. The water content indicated in wt. % refers to the amount of water present in the total weight of the agent. In a further especially particularly preferred embodiment, an agent according to the invention for temporarily changing the color is thus characterized by having, based on the total weight thereof, a water content between 20 and 60 wt. %, preferably between 24 and 54 wt. %, more preferably between 28 and 50 wt. %, and particularly preferably between 32 and 42 wt. %.

As a second component that is essential to the invention, the agents for temporarily changing the color comprise at least one color pigment (b). Within the meaning of the present invention, a pigment shall be understood to mean a color-imparting compound that has a solubility of less than 0.1 g/L at 20° C. in water.

The following method can be applied to determine the water solubility of the pigment. 0.1 g of the pigment is weighed in a beaker glass. A magnetic stir bar is added. Then distilled water (20° C.) is added to make 1 L. The mixture is stirred for one hour. If still undissolved components of the pigment are visible in this mixture after this period of time, the solubility of the pigment is less than 0.1 g/L.

The agents according to the invention are to be used to impart a temporary coloration. The emphasis in this regard is placed in particular on the generation of “metallic” effects. White pigments are therefore not covered by the definition of the color pigments. White pigments are achromatic inorganic pigments having a high refractive index (generally greater than 1.8), which are generally produced synthetically and are used primarily to generate optical whiteness in paints or as fillers, such as in plastic materials. White pigments, such as titanium dioxide or zinc dioxide, are explicitly not covered by the definition of a color pigment.

The color pigments are present in the agents in the form of small undissolved particles, which do not diffuse into the hair color, but deposit on the outer wall of the keratin fiber under the influence of the cationic cellulose(s) (c), where they are held by a film of the cationic cellulose (c).

Suitable color pigments can be of organic and/or inorganic origin.

The use of inorganic color pigments is particularly preferred in the method according to the invention given the excellent light, weather and/or temperature resistance thereof. The preferred average particle size of the, preferably inorganic, color pigments is 0.1 mm to 1 mm, more preferably 0.5 mm to 750 mm, and in particular 10 mm to 500 mm.

Preferred color pigments are selected from inorganic pigments, which may be of synthetic or natural origin. Inorganic color pigments of natural origin can be produced from chalk, red ocher, umbra, green earth, burnt sienna or graphite, for example. Furthermore, it is possible to use black pigments, such as iron oxide black, color pigments such as ultramarine or iron oxide red, and fluorescent or phosphorescent pigments as inorganic color pigments.

Colored metal oxides, metal hydroxides and metal oxide hydrates, mixed phase pigments, sulfurous silicates, silicates, metal sulfides, complex metal cyanides, metal sulfates, metal chromates and/or metal molybdates are particularly suitable. In particular, preferred color pigments are black iron oxide (Cl 77499), yellow iron oxide (Cl 77492), red and brown iron oxide (Cl 77491), manganese violet (Cl 77742), ultramarine (sodium aluminum sulfosilicates, Cl 77007, Pigment Blue 29), chromium oxide hydrate (CI 77289), iron blue (ferric ferrocyanide, CI 77510) and/or carmine (cochineal).

Particularly preferred color pigments according to the invention are colored pearlescing pigments. These are usually mica-based and can be coated with one or more metal oxides from the group consisting of titanium dioxide (CI 77891), black iron oxide (CI 77499), yellow iron oxide (CI 77492), red and brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Mica forms part of the phyllosilicates. The most important representatives of these silicates are muscovite, phlogopite, paragonite, biotite, lepidolite, and margarite. To produce the pearlescing pigments in combination with metal oxides, the mica, primarily muscovite or phlogopite, is coated with a metal oxide.

As an alternative to natural mica, it is also optionally possible to use synthetic mica coated with one or more metal oxides as the pearlescing pigment. Such suitable pearlescing pigments based on natural micas are described in the unexamined patent application WO 2005/065632, which is hereby explicitly included by reference. Particularly preferred pearlescing pigments are based on natural or synthetic mica and are coated with one or more of the aforementioned metal oxides. The color of the respective pigments can be varied by varying the layer thickness of the metal oxide or metal oxides.

In a further particularly preferred embodiment, an agent according to the invention is characterized by comprising, as the color pigment (b), at least one inorganic color pigment selected from the group consisting of colored metal oxides, metal hydroxides, metal oxide hydrates, silicates, metal sulfides, complex metal cyanides, metal sulfates, bronze pigments and/or colored pigments based on mica, which are coated with at least one metal oxide and/or a metal oxychloride.

In a further especially particularly preferred embodiment, an agent according to the invention is characterized by comprising, as the color pigment (b), at least one mica-based colored pigment, which is coated with one or more metal oxides from the group consisting of titanium dioxide (CI 77891), black iron oxide (CI 77499), yellow iron oxide (CI 77492), red and/or brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available, for example, under the trade names Rona®, Colorona®, Dichrona® and Timiron® from Merck, Ariabel® and Unipure® from Sensient, Prestige® from Eckart Cosmetic Colors, and Sunshine® from Sunstar.

Especially particularly preferred color pigments bearing the trade name Colorona® are, for example:

### Colorona Copper, Merck, MICA, Cl 77491 (IRON OXIDES)

### Colorona Passion Orange, Merck, Mica, Cl 77491 (Iron Oxides), Alumina

### Colorona Patina Silver, Merck, MICA, Cl 77499 (IRON OXIDES), Cl 77891 (TITANIUM DIOXIDE)

### Colorona RY, Merck, Cl 77891 (TITANIUM DIOXIDE), MICA, Cl 75470 (CARMINE)

### Colorona Oriental Beige, Merck, MICA, Cl 77891 (TITANIUM DIOXIDE), Cl 77491 (IRON OXIDES)

### Colorona Dark Blue, Merck, MICA, TITANIUM DIOXIDE, FERRIC FERROCYANIDE

### Colorona Chameleon, Merck, Cl 77491 (IRON OXIDES), MICA

### Colorona Aborigine Amber, Merck, MICA, Cl 77499 (IRON OXIDES), Cl 77891 (TITANIUM DIOXIDE)

### Colorona Blackstar Blue, Merck, Cl 77499 (IRON OXIDES), MICA

### Colorona Patagonian Purple, Merck, MICA, Cl 77491 (IRON OXIDES), Cl 77891 (TITANIUM DIOXIDE), Cl 77510 (FERRIC FERROCYANIDE)

### Colorona Red Brown, Merck, MICA, Cl 77491 (IRON OXIDES), Cl 77891 (TITANIUM DIOXIDE)

### Colorona Russet, Merck, Cl 77491 (TITANIUM DIOXIDE), MICA, Cl 77891 (IRON OXIDES)

### Colorona Imperial Red, Merck, MICA, TITANIUM DIOXIDE (Cl 77891), D&C RED NO. 30 (Cl 73360)

### Colorona Majestic Green, Merck, Cl 77891 (TITANIUM DIOXIDE), MICA, Cl 77288 (CHROMIUM OXIDE GREENS)

### Colorona Light Blue, Merck, MICA, TITANIUM DIOXIDE (Cl 77891), FERRIC FERROCYANIDE (Cl 77510)

### Colorona Red Gold, Merck, MICA, Cl 77891 (TITANIUM DIOXIDE), Cl 77491 (IRON)

### Colorona Gold Plus MP 25, Merck, MICA, TITANIUM DIOXIDE (Cl 77891), IRON OXIDES (Cl 77491)

### Colorona Carmine Red, Merck, MICA, TITANIUM DIOXIDE, CARMINE Colorona Blackstar Green, Merck, MICA, Cl 77499 (IRON OXIDES)

### Colorona Bordeaux, Merck, MICA, Cl 77491 (IRON OXIDES)

### Colorona Bronze, Merck, MICA, Cl 77491 (IRON OXIDES)

### Colorona Bronze Fine, Merck, MICA, Cl 77491 (IRON OXIDES)

### Colorona Fine Gold MP 20, Merck, MICA, Cl 77891 (TITANIUM DIOXIDE), Cl 77491 (IRON OXIDES)

### Colorona Sienna Fine, Merck, Cl 77491 (IRON OXIDES), MICA Colorona Sienna, Merck, MICA, Cl 77491 (IRON OXIDES)

Colorona Precious Gold, Merck, Mica, Cl 77891 (Titanium dioxide), Silica, Cl 77491 (Iron oxides), Tin oxide

### Colorona Sun Gold Sparkle MP 29, Merck, MICA, TITANIUM DIOXIDE, IRON OXIDES, MICA, Cl 77891, Cl 77491 (EU)

Colorona Mica Black, Merck, Cl 77499 (Iron oxides), Mica, Cl 77891 (Titanium dioxide)
Colorona Bright Gold, Merck, Mica, Cl 77891 (Titanium dioxide), Cl 77491 (Iron oxides)

### Colorona Blackstar Gold, Merck, MICA, Cl 77499 (IRON OXIDES)

Furthermore, particularly preferred color pigments bearing the trade name Unipure® are, for example:

### Unipure Red LC 381 EM, Sensient Cl 77491 (Iron Oxides), Silica

### Unipure Black LC 989 EM, Sensient, Cl 77499 (Iron Oxides), Silica

### Unipure Yellow LC 182 EM, Sensient, Cl 77492 (Iron Oxides), Silica

Depending on the degree of the change in color that is desired on the keratin fiber, the color pigment or color pigments (b) can be used in varying amounts. The more color pigment is used, the higher is the extent of the change in color in general. Starting at a certain usage amount, however, the adherence of the pigments to the keratin fiber approaches a limiting value, beyond which it is no longer possible to increase the extent of the change in color by further increasing the pigment amount used.

In this connection, it has been found that the use of the cationic celluloses (c), and in particular of the aforementioned preferred and particularly preferred representatives, on the keratin fiber allows a film to be formed, which causes the color pigments to adhere in particularly large amounts to the keratin fiber. The agents according to the invention can thus comprise the color pigments (b) in a total amount of 1.0 to 25.0 wt. %, preferably of 5.0 to 20.0 wt. %, more preferably of 7.0 to 18.0 wt. %, and particularly preferably of 8.5 to 15.5 wt. %.

In a further particularly preferred embodiment, an agent according to the invention is thus characterized by comprising, based on the total weight thereof, one or more color pigments (b) in a total amount of 1.0 to 25.0 wt. %, preferably of 5.0 to 20.0 wt. %, more preferably of 7.0 to 18.0 wt. %, and particularly preferably of 8.5 to 15.5 wt. %.

As a third component (c) that is essential to the invention, the agents for temporarily changing the color of keratin fiber comprise at least one cationic cellulose. Within the meaning of the present invention, cationic celluloses shall be understood to mean cellulose derivatives that have been synthetically modified by way of a reaction with one or more reagents such that these carry cationic charges or a large excess of cationic charges.

Cellulose is the main component of plant cell walls. Cellulose is unbranched and composed of between several hundred and ten thousand β-D-glucose molecules (β-1,4-glycosidic bond) or cellobiose units. Average molar masses are 50000 to 500000 g/mol. The average degree of polymerization (DP) of cellulose can be determined by various physical measurements. Most frequently, viscosity measurements are used to determine the degree of polymerization, which yield characteristic values, depending on the biological origin.

Quaternized hydroxyethyl celluloses have proven to be particularly suitable for achieving the object according to the invention. Hydroxyethyl celluloses are celluloses in which the hydroxy groups at the glucose units are substituted in one or more locations with (poly)hydroxyethyl groupings. Hydroxyethyl celluloses thus comprise at least one structural unit of formulas (a), (b) and/or (c).

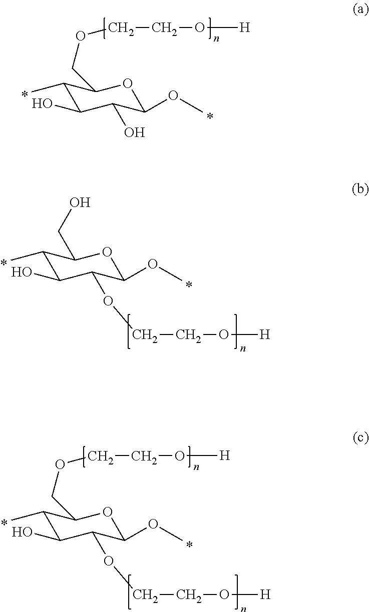

In the structural units (a), (b) and (c), each n, independently of one another, can take on an integer from 1 to 50, and preferably from 1 to 20. The positions marked with an asterisk each denote a linking site to the next glucose unit.

So as to introduce the cationic charge(s), the hydroxyethyl cellulose is now quaternized, which is to say a quaternary tetraalkylammonium group, which can optionally carry further substituents or heteroatoms, is introduced by way of a chemical reaction with a reagent.

In a further especially particularly preferred embodiment, an agent according to the invention is characterized by comprising at least one quaternized hydroxyethyl cellulose as the cationic cellulose (c).

It is particularly advantageous if the quaternized hydroxyethyl celluloses carry one or more structural units of formula (d),

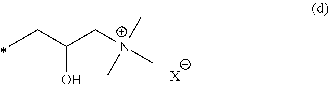

where Y can denote chloride, bromide, acetate or methyl sulfate. The position of group (d) denoted by the asterisk is preferably bound via a hydroxy group of the hydroxyethyl cellulose (which is to say the H-atom on a hydroxy group of the hydroxyethyl cellulose is replaced with a grouping (d)).

In a further especially particularly preferred embodiment, an agent according to the invention is characterized by comprising a cationic cellulose (c) having at least one structural unit of the general formula (I),

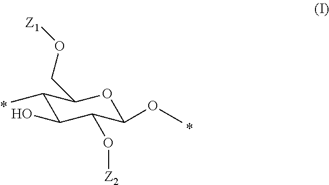

wherein
Z1 and/or Z2 either denote a hydrogen atom or a Y grouping;

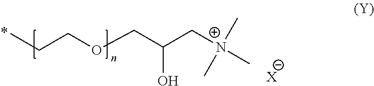

n independently in each of the Y groupings denotes an integer from 0 to 20;
Y denotes chloride, bromide, acetate or methyl sulfate,
with the proviso that at least one of the groupings Z1 and/or Z2 denotes a Y grouping.

Preferably, n denotes an integer of at least 1.

In a further especially particularly preferred embodiment, an agent according to the invention is characterized by comprising a cationic cellulose (c) having at least one structural unit of the general formula (I),

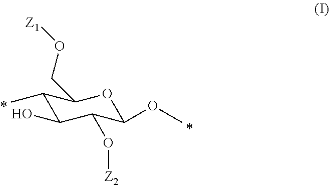

wherein
Z1 and/or Z2 either denote a hydrogen atom or a Y grouping;

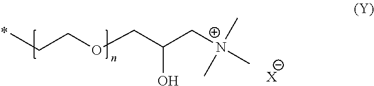

n independently in each of the Y groupings denotes an integer from 1 to 20;
Y denotes chloride, bromide, acetate or methyl sulfate,
with the proviso that at least one of the groupings Z1 and/or Z2 denotes a Y grouping.

The best results were achieved when Polyquaternium-10 was used as the cationic cellulose (c).

Polyquaternium-10 is also referred to as “Cellulose 2-hydroxyethyl 2-[2-hydroxy-3-(trimethylammonio)propoxy]ethyl 2-hydroxy-3-(trimethylammonio)propyl ether chloride” and has the CAS number 81859-24-7. The compound is commercially available, for example, under the trade name Antistatic 10 from 3V Sigma, and under the trade name JR 400 from Shanghai Jida meticulous Chemical Industry.

In a further especially particularly preferred embodiment, an agent according to the invention is characterized by comprising Polyquaternium-10 as the cationic cellulose (c).

Especially particularly preferred is an agent for temporarily changing the color of keratin fibers, and in particular of human hair, comprising, in an aqueous cosmetic carrier,
(a) at least 35.0 wt. % ethanol;
(b) at least one mica-based colored pigment, which is coated with one or more metal oxides from the group consisting of titanium dioxide (CI 77891), black iron oxide (CI 77499), yellow iron oxide (CI 77492), red and/or brown iron oxide (Cl 77491, CI 77499), manganese violet (Cl 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, Cl 77510); and
(c) Polyquaternium-10
wherein
the total amount of the lipids (d) present in the agent, based on the total weight of the agent, is less than 2.5% by weight.

Formulations according to the invention comprising Polyquaternium-10 have a very finely dispersed distribution of the color pigments (b) in the alcoholic-aqueous solution. The formulations can be sprayed very well, and the pigments can be evenly distributed on the keratin fibers, whereby a particularly homogeneous color result can be achieved. The agents according to the invention usually comprise the cationic cellulose or celluloses (c) in a total amount of 0.1 to 10.0 wt. %, preferably of 0.5 to 5.0 wt. %, more preferably of 0.8 to 3.0 wt. %, and particularly preferably of 1.4 to 2.6 wt. %. The quantity information in wt. % refers to the total amount of all cationic celluloses (c) relative to the total weight of the agent.

In a further particularly preferred embodiment, an agent according to the invention is characterized by comprising, based on the total weight thereof, one or more cationic celluloses (c) in a total amount of 0.1 to 10.0 wt. %, preferably of 0.5 to 5.0 wt. %, more preferably of 0.8 to 3.0 wt. %, and particularly preferably of 1.4 to 2.6 wt. %.

For optimal adhesive strength of the color pigments (b) to the keratin fibers, the amounts of pigments (b) and cationic cationic celluloses (c) used are advantageously matched to one another. If color pigments (b) and cationic celluloses (c) are used in a weight ratio of 1.0 to 6.0, the majority of the pigments is bound effectively by the cellulose film and thereby immobilized on the fiber. In other words, it is particularly advantageous to use color pigments (b) and cationic celluloses (c) at least in the same total amounts, or else to select usage amounts in which the total amount of the pigments (b) does not exceed the total amount of the cationic celluloses (b) by more than a factor of 6. At the indicated weight ratio of (b)/(c), the total amount of color pigments (b) present in the agent is present relative to the total amount of cationic celluloses (c) present in the agent.

In a further especially particularly preferred embodiment, an agent according to the invention is characterized in that the weight ratio of all color pigments (b) present in the agent to all cationic celluloses (c) present in the agent, which is to say the weight ratio (b)/(c), is 1.0 to 6.0, preferably 2.0 to 5.5, more preferably 2.5 to 5.0, and particularly preferably 3.0 to 4.5.

### Example

A temporary dye comprises

35.0 wt. % water;
(a) 40.0 wt. % ethanol;

### (b) 8.0 wt. % Colorona Bronze, Merck, MICA, Cl 77491 (IRON OXIDES);

### (c) 2.0 wt. % Polyquaternium-10;

further ingredients to make up to 100 wt. %;
weight ratio (b)/(c)=4.0

The hair mascara products known from the prior art generally comprise lipids; these lipids form a film on the keratin fibers, which protects the pigments against abrasion after being applied.

The key disadvantage of the lipids, however, is that these generate less advantageous haptics on the keratin fiber, manifested in particular in a sensation of roughness and a greasy feel of the hair. The keratin fibers appear to be weighed down, and also visually convey the impression of greasy hair.

So as to avoid this disadvantage, it is a characterizing and essential feature of the agents according to the invention that the total amount of the lipids (d) present in the agent, based on the total weight of the agent, is less than 2.5% by weight.

Within the meaning of the invention, “lipids” shall be understood to mean organic compounds having a solubility in water at room temperature (22° C.) and atmospheric pressure (760 mmHg) of less than 1 wt. %, and preferably of less than 0.1 wt. %. The definition of fat components explicitly covers only uncharged (which is to say non-ionic) compounds. Charged compounds, such as fatty acids and the salts thereof, are not considered to be lipids. Lipids within the meaning of the present invention comprise at least one saturated or unsaturated alkyl group having at least 12 carbon atoms. If the lipids comprise an unsaturated alkyl group, this may comprise one or more double bonds. The molecular weight of the fat components is no more than 5000 g/mol, preferably no more than 2500 g/mol, and particularly preferably no more than 1000 g/mol. The fat components are neither polyoxyalkylated nor polyglycerylated compounds, which is to say that fatty alcohols or fatty acids that are esterified or etherified with at least two oxyalkyl groups or with at least two glycerol units are not covered by the definition of the fats.

C₁₂-C₃₀ fatty alcohols are covered by the lipids (d). C₁₂-C₃₀ fatty alcohols are saturated, monounsaturated or polyunsaturated, linear or branched fatty alcohols having 12 to 30 carbon atoms. Examples of C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol). Examples of branched fatty alcohols are 2-octyldodecanol, 2-hexyldodecanol and/or 2-butyldodecanol.

C₁₂-C₃₀ fatty acid triglycerides are also covered by the lipids (d). A C₁₂-C₃₀ fatty acid triglyceride shall be understood to mean the triester of the trihydric alcohol glycerol with three equivalents of fatty acid. Both structurally identical and different fatty acids within a triglyceride molecule may be involved in the formations of esters. C₁₂-C₃₀ fatty acid diglycerides are also covered by the lipids. A C₁₂-C₃₀ fatty acid diglyceride shall be understood to mean the diester of the trihydric alcohol glycerol with two equivalents of fatty acid. Both structurally identical and different fatty acids within a diglyceride molecule may be involved in the formations of esters. C₁₂-C₃₀ fatty acid monoglycerides are also covered by the lipids. A C₁₂-C₃₀ fatty acid monoglyceride shall be understood to mean the monoester of the trihydric alcohol glycerol with one equivalent of fatty acid.

Lipids (d) also cover the diesters of one equivalent of ethylene glycol (1,2-ethanediol) with two equivalents of fatty acid (ethylene glycol di-fatty acid esters). Both structurally identical and different fatty acids may be involved in the ester bonds with the ethylene glycol.

Waxes are also covered by the lipids (d). Waxes shall be understood to mean the esters of C₁₂-C₃₀ fatty acids with C₁₂-C₃₀ fatty alcohols.

The lipids (d) also cover hydrocarbons comprising at least 12 carbon atoms. Hydrocarbons are exclusively compounds composed of the atoms carbon and hydrogen. Examples of hydrocarbons are mineral oils, liquid paraffin oils (such as paraffinum liquidum or paraffinum perliquidum), isoparaffin oils, semi-solid paraffin oils, paraffin waxes, solid paraffins (paraffinum solidum), vaseline and polydecenes. Silicones are not covered by the definition of the lipids.

In accordance with the invention is thus an agent for temporarily changing the color of keratin fibers, and in particular of human hair, comprising, in an aqueous cosmetic carrier,
(a) at least one aliphatic and/or aromatic alcohol having 2 to 8 carbon atoms;
(b) at least one color pigment; and
(c) at least one cationic cellulose;
wherein
the total amount of lipids (d) present in the agent from the group of the C₁₂-C₃₀ fatty alcohols, the C₁₂-C₃₀ fatty acid triglycerides, the C₁₂-C₃₀ fatty acid diglycerides, the C₁₂-C₃₀ fatty acid monoglycerides, the ethylene glycol di-fatty acid esters, the waxes and the hydrocarbons is thus less than 2.5 wt. %.

By using certain raw materials, it is possible, under certain circumstances, to introduce small amounts of lipids into the agents according to the invention. So as to weigh the hair down as little as possible, however, it is preferred to minimize the use of the lipids (d) to as great an extent as possible. It is thus preferred if the total amount of lipids (d) in the agent is less than 2.0 wt. %, preferably less than 1.5 wt. %, more preferably less than 0.5 wt. %, and particularly preferably less than 0.1 wt. %. The weight information is based on the total amounts of all lipids (a) relative to the total weight of the agent.

In a further especially particularly preferred embodiment, an agent according to the invention is thus characterized in that the total amount of all lipids (d) present in the agent, and in particular the lipids from the group consisting of the C₁₂-C₃₀ fatty alcohols, the C₁₂-C₃₀ fatty acid triglycerides, the C₁₂-C₃₀ fatty acid diglycerides, the C₁₂-C₃₀ fatty acid monoglycerides, the ethylene glycol di-fatty acid esters, the waxes and the hydrocarbons, is thus less than 2.0 wt. %, preferably less than 1.5 wt. %, more preferably less than 0.5 wt. %, and particularly preferably less than 0.1 wt.

Particularly preferred is also an agent for temporarily changing the color of keratin fibers, and in particular of human hair, comprising, in an aqueous cosmetic carrier,
(a) at least 35.0 wt. % ethanol;
(b) at least one color pigment; and
(c) at least one cationic cellulose;
wherein
the total amount of lipids (d) present in the agent from the group of the C₁₂-C₃₀ fatty alcohols, the C₁₂-C₃₀ fatty acid triglycerides, the C₁₂-C₃₀ fatty acid diglycerides, the C₁₂-C₃₀ fatty acid monoglycerides, the ethylene glycol di-fatty acid esters, the waxes and the hydrocarbons is thus less than 0.5 wt. %.

Especially particularly preferred is also an agent for temporarily changing the color of keratin fibers, and in particular of human hair, comprising, in an aqueous cosmetic carrier,
(a) at least 45.0 wt. % ethanol;
(b) at least one color pigment; and
(c) at least one cationic cellulose;
wherein
the total amount of lipids (d) present in the agent from the group of the C₁₂-C₃₀ fatty alcohols, the C₁₂-C₃₀ fatty acid triglycerides, the C₁₂-C₃₀ fatty acid diglycerides, the C₁₂-C₃₀ fatty acid monoglycerides, the ethylene glycol di-fatty acid esters, the waxes and the hydrocarbons is thus less than 0.5 wt. %.

The agents are provided in the form of aqueous-alcoholic preparations. Optionally, it is possible to additionally add a further surface-active substance to the agents, wherein these surface-active substances are referred to as surfactants or as emulsifiers, depending on the field of applications. The agents according to the invention preferably additionally comprise at least one non-ionic surfactant and/or one cationic surfactant. The use of anionic surfactants has not been found to be very advantageous.

The agents according to the invention can additionally comprise at least one non-ionic surfactant. Suitable non-ionic surfactants are alkyl polyglycosides and addition products of alkylene oxide to fatty alcohols and fatty acids, each having 2 to 30 moles ethylene oxide per mole of fatty alcohol or fatty acid. Preparations having good properties are likewise obtained if they include fatty acid esters of ethoxylated glycerol as non-ionic surfactants, which were reacted with at least 2 moles ethylene oxide.

The non-ionic surfactants are used in amounts of 0.1 to 45 wt. %, preferably of 1 to 30 wt. %, and especially particularly preferably of 1 to 15 wt. %, based on the total weight of the agent.

The agents according to the invention can additionally comprise at least one cationic surfactant. Cationic surfactants shall be understood to mean surfactants, which is to say surface-active compounds, each having one or more positive charges. Cationic surfactant exclusively have positive charges. These surfactants are usually composed of a hydrophobic part and a hydrophilic head group, wherein the hydrophobic part is generally composed of a hydrocarbon skeleton (for example, consisting of one or two linear or branched alkyl chains), and the positive charge or charges is or are localized in the hydrophilic head group. Examples of cationic surfactants are
quaternary ammonium compounds, which may carry one or two alkyl chains having a chain length of 8 to 28 carbon atoms as hydrophobic functional groups;
quaternary phosphonium salts, substituted with one or more alkyl chains having a chain length of 8 to 28 carbon atoms; or
tertiary sulfonium salts.

Moreover, the cationic charge may also be part of a heterocyclic ring (such as an imidazolium ring or a pyridinium ring) in the form of an onium structure.

In addition to the functional unit, which carries the cationic charge, the cationic surfactant may also comprise further uncharged functional groups, as is the case with esterquats, for example.

The cationic surfactants are used in amounts of 0.1 to 45 wt. %, preferably of 1 to 30 wt. %, and especially particularly preferably of 1 to 15 wt. %, based on the total weight of the agent.

The use of anionic surfactants has proven to be disadvantageous with respect to the abrasion resistance of the pigments on the keratin fibers. For this reason, it is preferred to use no anionic surfactants in the agents according to the invention.

Anionic surfactants are considered to be surface-active agents having exclusively anionic charges (neutralized by a corresponding counter-cation).

Examples of anionic surfactants are fatty acids, alkyl sulfates, alkyl ether sulfates and ether carboxylic acids having 12 to 20 carbon atoms in the alkyl group, and up to 16 glycol ether groups in the molecule.

In a further preferred embodiment, agents according to the invention are characterized in that the total amount of all anionic surfactants present in the agent is less than 2.5 wt., preferably less than 1.5 wt. %, more preferably less than 0.5 wt. %, and particularly preferably less than 0.1 wt., wherein all quantity information is based on the total weight of the agent.

The agents according to the invention can furthermore comprise at least one zwitterionic and/or amphoteric surfactant.

Suitable zwitterionic surfactants are betaines, N-alkyl-N,N-dimethylammonium glycinates, N-acyl-aminopropyl-N,N-methylammonium glycinates, and 2-alkyl-3-carboxymethyl-3-hydroxyethyl imidazolines. A preferred zwitterionic surfactant is known under the INCI name Cocamidopropyl Betaine.

Suitable amphoteric surfactants are N-alkyl glycines, N-alkyl propionic acids, N-alkyl aminobutyric acids, N-alkyl iminodipropionic acids, N-hydroxyethyl-N-alkylamidopropyl glycines, N-alkyl taurines, N-alkyl sarcosines, 2-alkyl aminopropionic acids and alkyl aminoacetic acids Particularly preferred amphoteric surfactants are N-coco alkyl aminopropionate, coco acyl aminoethylaminopropionate and C₁₂-C₁₈ acyl sarcosine.

The amphoteric and/or zwitterionic surfactants are used in amounts of 0.1 to 45 wt. %, preferably of 1 to 30 wt. %, and especially particularly preferably of 1 to 15 wt. %, based on the total weight of the agent.

One advantage of the agents according to the invention is that these can be formulated in a variety of forms. Very even color effects and rubbing-fast colorations can be achieved with an application by way of a sponge or by way of a small brush. However, it is also possible to formulate the agents according to the invention as sprays. In particular, the colorations obtained by way of the spray application are characterized by very high evenness.

Depending on the selected application form, the agents according to the invention are set to a certain viscosity. This is usually carried out by using one or more thickeners. There are no general restrictions with respect to these thickeners. It is possible to use both organic and purely inorganic thickeners.

Suitable thickeners are anionic synthetic polymers, cationic synthetic polymers, naturally occurring thickeners, such as non-ionic guar gums, scleroglucan gums or xanthan gums, gum arabic, ghatti gum, karaya gum, tragacanth gum, carrageenan gum, agar-agar, locust bean gum, pectins, alginates, starch fractions, and derivatives, such as amylose, amylopectin and dextrins, as well as cellulose derivatives (which are different from the celluloses according to the invention), such as methyl cellulose, carboxyalkyl celluloses, and hydroxyalkyl celluloses, non-ionic fully synthetic polymers, such as polyvinyl alcohol or polyvinylpyrrolidone, and inorganic thickeners, in particular phyllosilicates, such as bentonite, and in particular smectites, such as montmorrillonite or hectorite.

The viscosity of the agents can be set particularly easily and reproducibly by way of polysaccharides, and in particular polysaccharides from the group of the carboxy-C1-C6-alkyl celluloses, the hydroxy-C2-C8-alkyl celluloses, the alginic acids and/or xanthan gum.

By varying the polysaccharide amounts that are used, the agent can be formulated either as a gel for the brush or sponge application, or as a low-viscosity, sprayable solution. The other components of the recipe and/or the amounts thereof used do not have to be adapted. This is advantageous, in particular, for the production of the agents.

In a further particularly preferred embodiment, an agent according to the invention is thus characterized by additionally comprising at least one polysaccharide from the group of the carboxy-C₁-C₆-alkyl celluloses, the hydroxy-C₂-C₈-alkyl celluloses, the alginic acids and/or xanthan gum as the thickener.

In an especially particularly preferred embodiment, an agent according to the invention is characterized by additionally comprising at least one polysaccharide from the group of the hydroxy-C₂-C₈-alkyl celluloses as the thickener.

The thickener or thickeners can be used in the agents according to the invention in a total amount of 0.1 to 4.5 wt. %, preferably of 0.15 to 3.5 wt. %, and particularly preferably of 0.2 to 2.0 wt. %, based on the total weight of the agent.

For setting the pH value, the agents according to the invention can comprise one or more alkalizing agents. The alkalizing agents that can be used according to the invention to set the desired pH values can be selected from the group consisting of ammonia, alkanolamines, basic amino acids and inorganic alkalizing agents, such as alkaline earth and alkali metal hydroxides, alkaline earth and alkali metal metasilicates, alkaline earth and alkali metal phosphates, and alkaline earth and alkali metal hydrogen phosphates. For setting the pH value, the agents according to the invention can comprise one or more acids. Suitable acids are organic acids, for example, such as alpha-hydroxycarboxylic acid, or inorganic acids. The agents can furthermore comprise one or more non-ionic polymers.

Suitable non-ionogenic polymers are, for example:
vinylpyrrolidone/vinyl ester copolymers, as they are sold, for example, under the trade name Luviskol® (BASF). Luviskol® VA 64 and Luviskol® VA 73, each being vinylpyrrolidone/vinyl acetate copolymers, are likewise preferred non-ionic polymers.
starch and the derivative thereof, in particular starch ether, such as Structuret XL (National Starch), a multifunctional, salt-tolerant starch;
shellac;
polyvinylpyrrolidones, as they are sold, for example, under the designation Luviskol® (BASF).

Furthermore, the agents (V) and/or (F) can comprise one or more polymers from the group consisting of Polyquaternium-1, Polyquaternium-2, Polyquaternium-3, Polyquaternium-4, Polyquaternium-5, Polyquaternium-6, Polyquaternium-7, Polyquaternium-8, Polyquaternium-9, Polyquaternium-11, Polyquaternium-14, Polyquaternium-16, Polyquaternium-17, Polyquaternium-18, Polyquaternium-22, Polyquaternium-24, Polyquaternium-27, Polyquaternium-28, Polyquaternium-32, Polyquaternium-33, Polyquaternium-37, Polyquaternium-39, Polyquaternium-44, Polyquaternium-46, Polyquaternium-53, Polyquaternium-55, Polyquaternium-64, Polyquaternium-67, Polyquaternium-68, Polyquaternium-69 and/or Polyquaternium-86.

Moreover, the agents according to the invention can comprise further active ingredients, auxiliary agents and additives, for example linear cationic polymers, such as quaternized cellulose ethers, polysiloxanes comprising quaternary groups, dimethyldiallylammonium chloride polymers, dimethyldiallylammonium chloride/acrylamide copolymers, dimethylaminoethyl methacrylate/vinylpyrrolidinone copolymers quaternized with diethyl sulfate, vinylpyrrolidinonelimidazolinium methochloride copolymers, and quaternized polyvinyl alcohol; zwitterionic and amphoteric polymers; anionic polymers such as polyacrylic acids or cross-linked polyacrylic acids; structurants such as glucose, maleic acid and lactic acid; hair-conditioning compounds such as phospholipids, for example lecithin and kephalin, perfume oils, dimethyl isosorbide and cyclodextrins; fiber structure-enhancing active ingredients, in particular monosaccharides, disaccharides and oligosaccharides, such as glucose, galactose, fructose, levulose and lactose; dyes for dyeing the agent; anti-dandruff active ingredients such as piroctone olamine, zinc omadine and climbazole; amino acids and oligopeptides; animal- and/or plant-based protein hydrolyzates, and in the form of the fatty acid condensation products thereof, or optionally anionically or cationically modified derivatives; sunscreen agents and UV blockers; active ingredients such as panthenol, pantothenic acid, pantolactone, allantoin, pyrrolidinone carboxylic acids and the salts thereof, as well as bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonoles; ceramides or psudeoceramides; vitamins, provitamins and vitamin precursors; plant extracts; fats and waxes such as fatty alcohols, beeswax, montan wax and paraffins; swelling and penetration substances such as glycerol, propylene glycol monoethyl ether, carbonates, hydrogen carbonates, guanidines, ureas, as well as primary, secondary and tertiary phosphates; opacifying agents such as latex, styrene/PVP and styrene/acrylamide copolymers, and PEG-3 distearate; propellants such as propane-butane mixtures, N₂O, dimethyl ether, CO₂ and air.

A person skilled in the art will select these further substances in accordance with the desired properties of the agents. With respect to further optional components and the amounts of these components used, reference is expressly made to the relevant manuals known to the person skilled in the art. The respective additional active ingredients and auxiliary substances are preferably used in the agents according to the invention in quantities from 0.0001 to 25 wt. %, and in particular from 0.0005 to 15 wt. %, based on the total weight of the respective agents.

The products according to the invention can be formulated in the form of a gel, a spray, an aerosol or a pump mousse, for example. Depending on the form of application, these are therefore preferably loaded into a tube, a container, a bottle, a can, a pressurized container or a container comprising a pump spray applicator.

If the products are applied in spray form, the pigments can be applied particularly evenly onto the keratin fibers. The formulation as an aerosol or as a pump spray is therefore especially particularly preferred.

In the above-mentioned preferred embodiment, the product according to the invention comprises a pressurized container. Pressurized containers that may be used include vessels made of metal (aluminum, tinplate, tin), protected or shatterproof plastic, or glass, which are coated with plastic on the outside, in the selection of which pressure resistance, fracture strength, corrosion resistance, ease of filling, as well as aesthetic aspects, handling, printability and the like play a role. Special inside protective coatings can ensure corrosion resistance against the preparation present in the pressurized container.

If the product according to the invention is applied by way of a pressurized container, the agents additionally comprise at least one propellant from the group consisting of propane, propene, n-butane, iso-butane, iso-butene, n-pentane, pentene, iso-pentane, iso-pentene, air, nitrogen, argon, N₂O and/or CO₂.

Within this group, the permanent gases air, nitrogen, argon, N₂O and/or CO2 are preferred, with nitrogen, argon and/or CO₂ being especially particularly preferred.

Moreover, the agents according to the invention can also be applied in the form of a pump spray. Suitable containers comprising a pump or squeeze mechanism are commercially available from Rexam SMT or Seaquist, for example.

When used in the form of a pump spray or in the form of an aerosol spray, the user can spray the agents according to the invention directly onto the dry hair and in this way generate the desired temporary change in color.

The user can first style the hair, for example by way of combing, teasing or the use of a curling iron, and then spray on the agent according to the invention. Likewise, it is possible to spray on the agent according to the invention first, and after or during this process style the hair using the above-mentioned methods.

A second subject matter of the present invention is thus a method for temporarily changing the color and shape of hair, in which an agent of the first subject matter of the invention, which is formulated in the form of a pump spray or an aerosol spray, is sprayed onto the dry hair, and the hair is styled prior to or during the application.

What was said above with respect to the agents according to the invention applies, mutatis mutandis, with respect to preferred further embodiments of the method according to the invention.

### EXAMPLES

The following formulations were prepared; unless indicated otherwise, all information is in percent by weight (active substance).

1. Sponge application

| | % by weight |
|---|---|
| Stearamidopropyl dimethylamine | 1.3 |
| Lactic acid | 0.3 |
| Polyquaternium-10 | 2.0 |
| Glycerol | 2.0 |
| Cetyltrimethylammonium chloride | 0.45 |
| PEG-7 glyceryl cocoate | 2.0 |
| Hydroxyethyl cellulose | 0.2 |
| Colorona Precious Gold (Merck, Mica, CI 77891 | 8.5 |
| (titanium dioxide), silica, CI 77491 (iron oxides), tin | |
| oxide) | |
| Ethanol | 51 |
| Water | to make up to 100 |

The formulation was applied onto a dry strand of hair (Kerling dark blond) by way of a sponge. This yielded a uniformly dyed strand of hair having a golden sheen.

2. Brush application

| | % by weight |
|---|---|
| Stearamidopropyl dimethylamine | 1.3 |
| Lactic acid | 0.3 |
| Polyquaternium-10 | 2.0 |
| Glycerol | 2.0 |
| Cetyltrimethylammonium chloride | 0.45 |
| PEG-7 glyceryl cocoate | 2.0 |
| Hydroxyethyl cellulose | 2.0 |
| Colorona Precious Gold (Merck, Mica, CI 77891 | 8.5 |
| (titanium dioxide), silica, CI 77491 (iron oxides), tin | |
| oxide) | |
| Ethanol | 51 |
| Water | to make up to 100 |

The formulation was applied onto a dry strand of hair (Kerling dark blond) by way of a small brush. This yielded a uniformly dyed strand of hair having a golden sheen.

3. Spray application

| | % by weight |
|---|---|
| Stearamidopropyl dimethylamine | 1.3 |
| Lactic acid | 0.3 |
| Polyquaternium-10 | 2.0 |
| Glycerol | 2.0 |
| Cetyltrimethylammonium chloride | 0.45 |
| PEG-7 glyceryl cocoate | 2.0 |
| Colorona Precious Gold (Merck, Mica, CI 77891 | 8.5 |
| (titanium dioxide), silica, CI 77491 (iron oxides), tin | |
| oxide) | |
| Ethanol | 51 |
| Water | to make up to 100 |

The formulation was loaded into a pump spray atomizer and sprayed onto a dry strand of hair (Kerling dark blond). This yielded a uniformly dyed strand of hair having a golden sheen.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for temporarily changing the color of keratin fibers, and in particular of human hair, comprising, in an aqueous cosmetic carrier,
(a) at least one aliphatic and/or aromatic alcohol having 2 to 8 carbon atoms;
(b) at least one color pigment; and
(c) at least one cationic cellulose;
wherein the total amount of the lipids (d) present in the agent, based on the total weight of the agent, is less than 2.5% by weight.

2. The agent according to claim 1, wherein the one or more alcohols (a) are selected from the group consisting of ethanol, isopropanol, n-propanol, butanol, n-pentanol, 1,2-propanediol, 1,3-propandiol, 1,3-butanediol, 1,4-butanediol, 1,2-hexanediol, 1,6-hexanediol, glycerol, benzyl alcohol, phenoxyethanol and phenylethyl alcohol.

3. The agent according to claim 1, wherein the one or more alcohols (a) are included in a total amount of at least 10.0 wt. %.

4. The agent according to claim 3, wherein the one or more alcohols (a) are included in a total amount of at least 50.0 wt.

5. The agent according to claim 1, comprising, based on the total weight thereof, at least 15.0 wt. % ethanol.

6. The agent according to claim 1, comprising, based on the total weight thereof, 1,2-propanediol and/or glycerol in a total amount of 0.1 to 7.0 wt. %

7. The agent according to claim 1, including, based on the total weight thereof, a water content between 20 and 60 wt. %.

8. The agent according to claim 1, including, as the color pigment (b), at least one inorganic color pigment selected from the group consisting of colored metal oxides, metal hydroxides, metal oxide hydrates, silicates, metal sulfides, complex metal cyanides, metal sulfates, bronze pigments and colored pigments based on mica, which are coated with at least one metal oxide and/or one metal oxychloride.

9. The agent according to claim 1, including, as the color pigment (b), at least one mica-based colored pigment, which is coated with one or more metal oxides selected from the group consisting of titanium dioxide (CI 77891), black iron oxide (CI 77499), yellow iron oxide (CI 77492), red iron oxide (CI 77491), brown iron oxide (CI 77499), manganese violet (Cl 77742), ultramarine (sodium aluminum sulfosilicates, CI 77007, Pigment Blue 29), chromium oxide hydrate (CI 77289), chromium oxide (Cl 77288) and iron blue (ferric ferrocyanide, CI 77510).

10. The agent according to claim 1, including, based on the total weight thereof, one or more of the color pigments (b) in a total amount of 1.0 to 25.0 wt. %.

11. The agent according to claim 1, including at least one quaternized hydroxyethyl cellulose as the cationic cellulose (c).

12. The agent according to claim 1, wherein the cationic cellulose (c) includes at least one structural unit of the general formula (I), 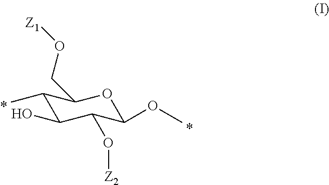
wherein
Z1 and/or Z2 either denote a hydrogen atom or a Y grouping; 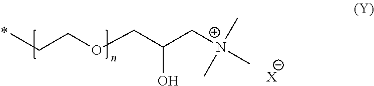
n independently in each of the Y groupings denotes an integer from 0 to 20;
X⁻ denotes chloride, bromide, acetate or methyl sulfate,
with the proviso that at least one of the groupings Z1 and Z2 denotes a Y grouping.

13. The agent according to claim 1, wherein the cationic cellulose (c) includes Polyquaternium-10.

14. The agent according to claim 1, wherein the one or more cationic celluloses (c) are included in a total amount of 0.1 to 10.0 wt. %.

15. The agent according to claim 1, wherein a weight ratio of all color pigments (b) present in the agent to all cationic celluloses (c) present in the agent, which is to say the weight ratio (b)/(c), is 1.0 to 6.0.

16. A method for temporarily changing the color and shape of hair, including
spraying an agent according to claim 1, which is formulated in the form of a pump spray or an aerosol spray, onto the dry hair, and
styling the hair prior to or during the application.
